(19) United States
(12) Patent Application Publication
(10) Pub. No.: US 2018/0161262 A1
(43) Pub. Date: Jun. 14, 2018

(54) Cosmetic Method and Dermatological Topical Agent for Use Therein, Migration Imparting Agent, and Method for Screening Components for Use in Cosmetic Method for Improving Skin State

(71) Applicant: ROHTO PHARMACEUTICAL CO., LTD., Osaka (JP)

(72) Inventors: Tomohiro Tsuda, Osaka (JP); Masayo Yumoto, Osaka (JP)

(73) Assignee: ROHTO PHARMACEUTICAL CO., LTD., Osaka (JP)

(21) Appl. No.: 15/577,552

(22) PCT Filed: May 26, 2016
(86) PCT No.: PCT/JP2016/065560

§ 371 Date: Nov. 28, 2017

(30) Foreign Application Priority Data
May 29, 2015 (JP) 2015-109656
Dec. 1, 2015 (JP) 2015-234603

(51) Int. Cl. A61K 8/65; A61Q 19/00; A61K 8/64; A61K 8/34; G01N 33/50

(52) U.S. Cl. CPC A61K 8/65; A61Q 19/00; A61K 8/64; A61K 2800/592; G01N 33/502; G01N 33/5073; A61K 8/345

(57) ABSTRACT

The present invention relates to a cosmetic method comprising applying a migration imparting agent for adipose-derived stem cells to the skin, thereby allowing adipose-derived stem cells to migrate to the dermal layer and improving a skin condition. The present invention also relates to an external skin preparation for use in the cosmetic method, the external skin preparation comprising a migration imparting agent allowing adipose-derived stem cells to migrate to the dermal layer by application to the skin. The present invention further relates to a migration imparting agent for adipose-derived stem cells, comprising at least one of a polyol compound and a hydrophilic biomaterial. The present invention further relates to a method of screening for a component for use in a cosmetic method for improving a skin condition, comprising evaluating migratory activity of adipose-derived stem cells by using a prepared medium containing a sample.

### TECHNICAL FIELD

The present invention relates to a cosmetic method of improving a skin condition, a dermatological topical agent (an external skin preparation) for use therein, a migration imparting agent, and a method of screening for a component for use in a cosmetic method for improving a skin state (a skin condition).

### BACKGROUND ART

In recent years, expectations for regenerative medicine which repairs or regenerates injured tissues or organs in human bodies by using stem cells or cells derived from stem cells have been raised, and research on stem cells is rapidly advancing. The stem cells are cells having both of the ability to differentiate into diverse types according to the need (multilineage potential) and the ability to maintain the multilineage potential after cell division (self-renewal potential), and ES cells (embryonic stem cells), iPS cells (induced pluripotent stem cells), and somatic stem cells (adult stem cells) are known.

Among these stem cells, ES cells employ fertilized eggs and therefore present many problems to be overcome, such as ethical problems. Also, the iPS cells employ human somatic cells reprogrammed into stem cells and still require many clinical studies.

By contrast, it is highly likely that the somatic stem cells can be useful in regenerative medicine by utilizing the original functions of the somatic stem cells possessed by living bodies, and practical research is most advancing as compared with other stein cells. As a consequence of such research, for example, in Patent Literature 1, use of a specific substance such as Tiliaceae plant extracts or Paeonia suffruticosa Andrews root bark extracts as an attractant for mesenchymal stem cells is proposed. According to this proposition, the possibility is indicated that mesenchymal stem cells in blood circulating in the body by blood flow can be attracted to accumulate in a specific tissue.

Also, in Patent Literature 2, it is suggested that a specific substance such as extracts of Phyllanthus emblica or Emblica officinalis, which is a medium-sized deciduous tree, is effective for the increased production of PDGF-BB (platelet-derived growth factor) which contributes to the stabilization of mesenchymal stem cells, and the stabilization of stem cells is attempted by using this.

### CITATION LIST

### Patent Literature

Patent Literature 1: Japanese Unexamined Patent Publication No. 2011-251925

Patent Literature 2: Japanese Unexamined Patent Publication No. 2013-1669

### SUMMARY OF INVENTION

### Problems to be Solved by the Invention

While these propositions lead to regenerative medicine, cosmetic effects such as an antiaging effect on the skin are expected. However, in Patent Literature 1 described above, the targeted mesenchymal stem cells are typically bone marrow-derived mesenchymal stem cells contained in blood and are located far away from the dermal layer; and in Patent Literature 2 described above, though stating that the specific substance is effective for the increased production of PDGF-BB which contributes to the stabilization of stem cells, it is uncertain whether or not cosmetic effects are obtained because it does not have direct influence on stem cells.

The present invention has been made in light of these situations, and an object thereof is to provide a cosmetic method using, particularly, adipose-derived stem cells present in the fat layer right below the dermal layer among somatic stem cells, an external skin preparation for use therein, a migration imparting agent, and a method of screening for a component for use in a cosmetic method for improving a skin condition.

### Means for Solving the Problems

To attain the object described above, a first aspect of the present invention is a cosmetic method comprising applying a migration imparting agent for adipose-derived stem cells (hereinafter, also simply referred to as a “migration imparting agent”) to the skin, thereby allowing adipose-derived stem cells to migrate to the dermal layer and improving a skin condition.

A second aspect of the present invention is particularly the cosmetic method, wherein the migration imparting agent is at least one of a polyol compound and a hydrophilic biomaterial.

A third aspect of the present invention is particularly the cosmetic method, wherein the polyol compound is at least one polyol having two or more hydroxy groups on an alkyl group having 3 to 6 carbon atoms, and a fourth aspect is the cosmetic method, wherein the polyol having two or more hydroxy groups on an alkyl group having 3 to 6 carbon atoms is at least one selected from the group consisting of dipropylene glycol, 1,2-butylene glycol, 1,3-butylene glycol, propylene glycol, 1,3-propanediol, 1,2-pentanediol, 1,2-hexanediol, ethylene glycol and diethylene glycol.

A fifth aspect of the present invention is particularly the cosmetic method, wherein the hydrophilic biomaterial is at least one selected from the group consisting of monosaccharides, disaccharides, oligosaccharides, polysaccharides, mucopolysaccharides, collagens, oligopeptides and polypeptides, and a sixth aspect is the cosmetic method, wherein the oligopeptides are at least one selected from the group consisting of dipeptides, tripeptides, tetrapeptides and derivatives thereof.

A seventh aspect of the present invention is particularly the cosmetic method, wherein the migration imparting agent is at least one selected from the group consisting of collagen, copper tripeptide-1, caprooyl tetrapeptide-3, and dipropylene glycol, and an eighth aspect is the cosmetic method, wherein the migration imparting agent is a combination of collagen, copper tripeptide-1 and caprooyl tetrapeptide-3.

A ninth aspect of the present invention is an external skin preparation for use in the cosmetic method according to any of the first to eighth aspects, the external skin preparation comprising a migration imparting agent allowing adipose-derived stem cells to migrate to the dermal layer by application to the skin.

A tenth aspect of the present invention is a migration imparting agent for adipose-derived stein cells, the migration imparting agent comprising at least one of a polyol compound and a hydrophilic biomaterial.

An eleventh aspect of the present invention is a method of screening for a component for use in a cosmetic method for improving a skin condition, the screening method comprising evaluating migratory activity of adipose-derived stem cells by using a prepared medium containing a sample.

A twelfth aspect of the present invention is particularly the method of screening for a component for use in a cosmetic method for improving a skin condition, wherein the skin condition is a condition of the non-wound skin.

A thirteenth aspect of the present invention is a cosmetic method comprising allowing fibroblasts and adipose-derived stem cells to coexist in the dermal layer, thereby improving a skin condition.

A fourteenth aspect of the present invention is particularly the cosmetic method, wherein the fibroblasts and the adipose-derived stem cells are allowed to coexist by migration, implantation, or penetration of the adipose-derived stem cells.

Specifically, the cosmetic method of the present invention is based on the idea that since, particularly, adipose-derived stem cells among somatic stem cells are abundantly present in the fat layer right below the dermal layer, they may be encouraged to contribute to improvement in skin condition in the dermal layer, and involves applying a specific migration imparting agent to the skin, thereby allowing stem cells in the fat layer to migrate to the dermal layer and growing or activating the adipose-derived stem cells in the dermal layer to improve a skin condition.

More specifically, it is known that in the case where injury or the like occurs in a tissue in a living body, stem cells generally move to the injured site, differentiate into the damaged cells or suppress the damage, and exert the effect of repairing this. Such a behavior in which cells move toward a specific site is called “migration (or homing)”. The present inventors have focused on such a property of migration of stem cells and gained the idea that if a migration imparting agent, which is a substance having the effect of allowing stem cells to migrate or promoting migration, is applied to the skin, stem cells can migrate from the fat layer below the dermal layer, and cosmetic effects on the skin can be enhanced by utilizing these adipose-derived stem cells, reaching the present invention as a result of conducting a series of studies.

The present invention mentioned above can also be interpreted as each of the following aspects:

[1-1] An external skin preparation for improvement in skin condition, the external skin preparation comprising a migration imparting agent for adipose-derived stem cells.
[1-2] The external skin preparation according to [1-1], wherein the improvement in skin condition is based on migration of adipose-derived stem cells to the dermal layer.
[1-3] The external skin preparation according to [1-1] or [1-2], wherein the migration imparting agent is at least one of a polyol compound and a hydrophilic biomaterial.
[1-4] The external skin preparation according to [1-3], wherein the polyol compound is at least one polyol having two or more hydroxy groups on an alkyl group having 3 to 6 carbon atoms.
[1-5] The external skin preparation according to [1-4], wherein the polyol having two or more hydroxy groups on an alkyl group having 3 to 6 carbon atoms is at least one selected from the group consisting of dipropylene glycol, 1,2-butylene glycol, 1,3-butylene glycol, propylene glycol, 1,3-propanediol, 1,2-pentanediol, 1,2-hexanediol, ethylene glycol and diethylene glycol.
[1-6] The external skin preparation according to any of [1-3] to [1-5], wherein the hydrophilic biomaterial is at least one selected from the group consisting of monosaccharides, disaccharides, oligosaccharides, polysaccharides, mucopolysaccharides, collagens, oligopeptides and polypeptides.
[1-7] The external skin preparation according to [1-6], wherein the oligopeptides are at least one selected from the group consisting of dipeptides, tripeptides, tetrapeptides and derivatives thereof.
[1-8] The external skin preparation according to any of [1-1] to [1-7], wherein the migration imparting agent is at least one selected from the group consisting of collagen, copper tripeptide-1, caprooyl tetrapeptide-3, and dipropylene glycol.
[1-9] The external skin preparation according to any of [1-1] to [1-8], wherein the migration imparting agent is a combination of collagen, copper tripeptide-1 and caprooyl tetrapeptide-3.
[1-10] The external skin preparation according to [1-9], wherein the migration imparting agent contains the collagen, the copper tripeptide-1 and the caprooyl tetrapeptide-3 at a ratio of 1:0.01 to 10:0.01 to 10 based on weight.

The present invention can also be interpreted as each of the following aspects:

[2-1] An external skin preparation for use in improvement in skin condition, the external skin preparation comprising a migration imparting agent for adipose-derived stem cells.
[2-2] The external skin preparation for use in improvement in skin condition according to [2-1], wherein the improvement in skin condition is based on migration of adipose-derived stem cells to the dermal layer.
[2-3] The external skin preparation for use in improvement in skin condition according to [2-1] or [2-2], wherein the migration imparting agent is at least one of a polyol compound and a hydrophilic biomaterial.
[2-4] The external skin preparation for use in improvement in skin condition according to [2-3], wherein the polyol compound is at least one polyol having two or more hydroxy groups on an alkyl group having 3 to 6 carbon atoms.
[2-5] The external skin preparation for use in improvement in skin condition according to [2-4], wherein the polyol having two or more hydroxy groups on an alkyl group having 3 to 6 carbon atoms is at least one selected from the group consisting of dipropylene glycol, 1,2-butylene glycol, 1,3-butylene glycol, propylene glycol, 1,3-propanediol, 1,2-pentanediol, 1,2-hexanediol, ethylene glycol and diethylene glycol.
[2-6] The external skin preparation for use in improvement in skin condition according to any of [2-3] to [2-5], wherein the hydrophilic biomaterial is at least one selected from the group consisting of monosaccharides, disaccharides, oligosaccharides, polysaccharides, mucopolysaccharides, collagens, oligopeptides and polypeptides.
[2-7] The external skin preparation for use in improvement in skin condition according to [2-6], wherein the oligopeptides are at least one selected from the group consisting of dipeptides, tripeptides, tetrapeptides and derivatives thereof.
[2-8] The external skin preparation for use in improvement in skin condition according to any of [2-1] to [2-7], wherein the migration imparting agent is at least one selected from the group consisting of collagen, copper tripeptide-1, caprooyl tetrapeptide-3, and dipropylene glycol.
[2-9] The external skin preparation for use in improvement in skin condition according to any of [2-1] to [2-8], wherein the migration imparting agent is a combination of collagen, copper tripeptide-1 and caprooyl tetrapeptide-3.
[2-10] The external skin preparation for use in improvement in skin condition according to [2-9], wherein the migration imparting agent contains the collagen, the copper tripeptide-1 and the caprooyl tetrapeptide-3 at a ratio of 1:0.01 to 10:0.01 to 10 based on weight.

The present invention can also be interpreted as each of the following aspects:

[3-1] Use of a migration imparting agent for adipose-derived stem cells for production of an external skin preparation for use in improvement in skin condition.
[3-2] The use according to [3-1], wherein the improvement in skin condition is based on migration of adipose-derived stem cells to the dermal layer.
[3-3] The use according to [3-1] or [3-2], wherein the migration imparting agent is at least one of a polyol compound and a hydrophilic biomaterial.
[3-4] The use according to [3-3], wherein the polyol compound is at least one polyol having two or more hydroxy groups on an alkyl group having 3 to 6 carbon atoms.
[3-5] The use according to [3-4], wherein the polyol having two or more hydroxy groups on an alkyl group having 3 to 6 carbon atoms is at least one selected from the group consisting of dipropylene glycol, 1,2-butylene glycol, 1,3-butylene glycol, propylene glycol, 1,3-propanediol, 1,2-pentanediol, 1,2-hexanediol, ethylene glycol and diethylene glycol.
[3-6] The use according to any of [3-3] to [3-5], wherein the hydrophilic biomaterial is at least one selected from the group consisting of monosaccharides, disaccharides, oligosaccharides, polysaccharides, mucopolysaccharides, collagens, oligopeptides and polypeptides.
[3-7] The use according to [3-6], wherein the oligopeptides are at least one selected from the group consisting of dipeptides, tripeptides, tetrapeptides and derivatives thereof.
[3-8] The use according to any of [3-1] to [3-7], wherein the migration imparting agent is at least one selected from the group consisting of collagen, copper tripeptide-1, caprooyl tetrapeptide-3, and dipropylene glycol.
[3-9] The use according to any of [3-1] to [3-8], wherein the migration imparting agent is a combination of collagen, copper tripeptide-1 and caprooyl tetrapeptide-3.
[3-10] The use according to [3-9], wherein the migration imparting agent contains the collagen, the copper tripeptide-1 and the caprooyl tetrapeptide-3 at a ratio of 1:0.01 to 10:0.01 to 10 based on weight.

The present invention can also be interpreted as each of the following aspects:

[4-1] An agent for use in migration of adipose-derived stem cells or in promotion of migration of adipose-derived stem cells, the agent comprising at least one of a polyol compound and a hydrophilic biomaterial.
[4-2] Use of at least one of a polyol compound and a hydrophilic biomaterial in production of an agent for use in migration of adipose-derived stem cells or in promotion of migration of adipose-derived stem cells.

The present invention can also be interpreted as each of the following aspects:

[5-1] A method of screening for a component improving a condition of the non-wound skin, the screening method comprising:

respectively providing a prepared medium containing a sample and a standard medium not containing the sample;

culturing adipose-derived stem cells for a predetermined period in Transwell by using the prepared medium or the standard medium; and

selecting the component improving a condition of the non-wound skin by using, as an index, higher migratory activity of the adipose-derived stem cells when the prepared medium is used than the migratory activity of the adipose-derived stem cells when the standard medium is used.

[5-2] A method of screening for a component improving a condition of the non-wound skin, the screening method comprising:

providing a three-dimensional skin model in which a labeled adipose-derived stem cell layer, a dermal model layer and an epidermal keratinocyte layer are stacked in this order;

applying a sample to the skin model, followed by culture for a predetermined period;

measuring the number of adipose-derived stem cells that have migrated to the dermal model layer and/or the epidermal keratinocyte layer by using the label in the skin model after the culture; and selecting the component improving a condition of the non-wound skin by using, as an index, the number of adipose-derived stem cells that have migrated to the dermal model layer and/or the epidermal keratinocyte layer.

### Effects of the Invention

According to the cosmetic method of the present invention, adipose-derived stem cells present in the fat layer or the dermal layer can be grown or activated by applying a migration imparting agent, which is a substance having the effect of allowing adipose-derived stem cells to migrate or promoting migration, to the skin. Furthermore, stem cells in the dermal layer can be allowed to exist more abundantly than the normal state by allowing the activated adipose-derived stem cells in the fat layer to migrate to the dermal layer. The adipose-derived stem cells that have migrated secrete, in themselves, factors associated with the construction of extracellular matrix (hereinafter, also abbreviated to “ECM”) in the dermal layer and also activate fibroblasts in the dermal layer. For example, damage on fibroblasts and the like in the dermal layer is rapidly repaired, or the fibroblasts are activated. Thus, cellular senescence or injury which impairs a skin condition is ameliorated, and skin elasticity and firmness are kept good so that excellent cosmetic effects are obtained.

In the cosmetic method of the present invention, particularly, in the case where the migration imparting agent is at least one of a polyol compound and a hydrophilic biomaterial, a migration imparting effect (at least one effect of the effect of allowing cells to migrate and the effect of promoting migration) on adipose-derived stem cells is especially high, and excellent cosmetic effects can be obtained.

Also, in the case where the polyol compound is particularly at least one polyol having two or more hydroxy groups on an alkyl group having 3 to 6 carbon atoms, and in the case where the polyol is at least one selected from the group consisting of dipropylene glycol, 1,2-butylene glycol, 1,3-butylene glycol, propylene glycol, 1,3-propanediol, 1,2-pentanediol, 1,2-hexanediol, ethylene glycol and diethylene glycol, an especially excellent migration imparting effect is obtained.

Furthermore, in the case where the hydrophilic biomaterial is at least one selected from the group consisting of monosaccharides, disaccharides, oligosaccharides, polysaccharides, mucopolysaccharides, collagens, oligopeptides and polypeptides, or in the case where the oligopeptides are at least one selected from the group consisting of dipeptides, tripeptides, tetrapeptides and derivatives thereof, a much better migration imparting effect is obtained, which is preferable.

According to the external skin preparation of the present invention, by applying this to the skin, a migration imparting effect is exerted on adipose-derived stem cells in the fat layer in the inside of the skin, whereby movement of stem cells to the skin surface side, i.e., the dermal layer, becomes possible, and stem cells in the dermal layer can be grown or activated. Furthermore, a skin condition can be improved, for example, by activating fibroblasts which impart elasticity and firmness to the skin, through the action of these stem cells.

The migration imparting agent of the present invention can be suitably used in the external skin preparation and the like.

According to the method of the present invention for screening for a component for use in a cosmetic method for improving a skin condition, a component capable of contributing to improvement in skin condition can be efficiently screened for. Furthermore, the screening method can be suitably used, for example, in the case where the skin condition is a condition of the non-wound skin.

The cosmetic method of the present invention comprising allowing fibroblasts and adipose-derived stem cells to coexist in the dermal layer is not necessarily required to apply the migration imparting agent as described above, and an excellent skin condition improving effect, as in the case of applying the migration imparting agent, can be obtained by allowing the adipose-derived stem cells to coexist in the dermal layer, for example, by an operation such as direct implantation of the adipose-derived stem cells to the dermal layer, or penetration.

### BRIEF DESCRIPTION OF DRAWINGS

FIG. 1 is a diagram illustrating an apparatus that is used in a scaffold migration test.

FIG. 2 is a fluorescence microscope photograph of a scaffold section of Example 6.

FIG. 3 is a fluorescence microscope photograph of a scaffold section of Comparative Example 2.

FIG. 4 is a graph chart in which the amount of type 1 collagen produced was compared between human adipose-derived stem cells and human dermal fibroblasts.

FIG. 5 is a graph chart in which the amount of MMP1 produced was compared between human adipose-derived stem cells and human dermal fibroblasts.

FIG. 6 is a graph chart in which MMP1 activity was compared between human adipose-derived stem cells and human dermal fibroblasts.

FIG. 7 is a graph chart in which the amount of hyaluronic acid produced was compared between human adipose-derived stem cells and human dermal fibroblasts.

FIG. 8 is a graph chart in which the amount of TIMP1 produced was compared between human adipose-derived stem cells and human dermal fibroblasts.

FIG. 9 is a graph chart showing the influence of coexistence of human adipose-derived stem cells and human dermal fibroblasts on the amount of TIMP1 produced in the human dermal fibroblasts.

FIG. 10 is a graph chart showing the influence of coexistence of human adipose-derived stem cells and human dermal fibroblasts on the amount of hyaluronic acid produced in the human dermal fibroblasts.

FIG. 11 is a graph chart showing the influence of coexistence of human adipose-derived stem cells and human dermal fibroblasts on the amount of type I collagen produced in the human dermal fibroblasts.

### EMBODIMENTS FOR CARRYING OUT THE INVENTION

Next, embodiments of the present invention will be described in detail. However, the present invention is not limited by the embodiments given below.

In one embodiment, the cosmetic method of the present invention comprises applying a migration imparting agent to the skin, thereby allowing adipose-derived stem cells to migrate to the dermal layer and improving a skin condition. The cosmetic method of the present invention may be carried out in a form for purely cosmetic purposes (form not involving medical practice on humans).

The adipose-derived stem cells are, as already mentioned, stem cells abundantly present in the fat layer in a so-called subcutaneous tissue below the dermal layer of the skin, and possess self-renewal potential and multilineage potential, as with other stem cells. Particularly, a feature of the adipose-derived stem cell is to possess high multilineage potential as compared with bone marrow-derived stem cells, which are also somatic stem cells. In the present specification, the adipose-derived stem cells usually mean mammalian adipose-derived stem cells, preferably human, pig, bovine, dog, or cat adipose-derived stem cells, particularly preferably human adipose-derived stem cells.

The migration imparting agent used in the present invention refers to a substance having at least one effect of the effect of allowing adipose-derived stem cells to migrate to a location where this migration imparting agent is present, and the effect of promoting migration of adipose-derived stem cells themselves, and is not particularly limited as long as having the effect. Examples thereof include plant-derived substances, animal-derived substances (non-plant-derived substances), and synthetic products, and among them, particularly, a polyol compound or a hydrophilic biomaterial is preferably used. A non-plant-derived one tends to be preferably used. These migration imparting agents may be used singly or in combinations of two or more.

Examples of the polyol compound include polyols having two or more hydroxy groups on an alkyl group having 3 to 6 carbon atoms (C3 to C6 alkyl group). More specifically, examples thereof include dipropylene glycol, 1,2-butylene glycol, 1,3-butylene glycol, 1,4-butanediol, 1,3-butanediol, propylene glycol, isopropylene glycol, 1,3-propanediol, isoprene glycol, 1,2-pentanediol, hexylene glycol, 1,2-hexanediol, 1,6-hexanediol, glycerin, diglycerin, triglycerin, xylitol, galactitol, sorbitol, mannitol, ethylene glycol and diethylene glycol, and among them, dipropylene glycol is preferably used. These polyol compound may be used singly or in combinations of two or more.

In the present invention, in the case of using the polyol compound as the migration imparting agent and preparing this as an external skin preparation, the content of the polyol compound is, for example, preferably 0.000001% by weight or more, more preferably 0.00001% by weight or more, still more preferably 0.0001% by weight or more, further preferably 0.001% by weight or more, with respect to the total weight of the external skin preparation from the viewpoint of exerting the effect of the present invention. Also, it is, for example, preferably 25% by weight or less, more preferably 15% by weight or less, further preferably 10% by weight or less, most preferably 5% by weight or less, with respect to the total weight of the external skin preparation from the viewpoint of reducing an unpleasant feeling such as stickiness upon use.

In the present invention, examples of the hydrophilic biomaterial that can be used as the migration imparting agent include monosaccharides, disaccharides, oligosaccharides, polysaccharides, mucopolysaccharides, collagens, oligopeptides, and polypeptides, and among them, collagens and oligopeptides are especially preferable. These hydrophilic biomaterials may also be used singly or in combinations of two or more.

Although the collagens are not particularly limited by the origin of a raw material (collagen), ones derived from cnidarians (e.g., jellyfish), echinoderms (e.g., sea cucumber), fish, cattle, pigs, chickens, and the like are preferable because stable supply is possible. The collagens according to the present invention also include low-molecular collagen prepared by partially degrading collagen (natural collagen derived from fish or the like) with an enzyme (e.g., protein hydrolase such as collagenase, pepsin, or trypsin) and/or an acid or an alkali.

Specific examples of such low-molecular collagen include low-molecular collagen prepared by adding water to natural collagen, extracting collagen by heating or pressurization and heating, and treating this collagen with protein hydrolase. The type of the enzyme is not particularly limited, and, for example, neutral protease, alkaline protease, acidic protease, or an enzyme preparation containing the same can be used. The obtained enzymatic degradation product is subjected to reverse osmosis membrane treatment to recover a concentrate. The concentrate (collagen peptide) may be used as it is or may be used after being appropriately dried and pulverized.

Commercially available collagen (e.g., “MARINGEN SP-03(PF)” manufactured by Nitta Gelatin Inc.) can also be used as a starting material for the collagen peptide. Alternatively, commercially available low-molecular collagen can also be used. Examples of the commercially available product include “PROMOIS W-32”, “PROMOIS W-32LS”, “PROMOIS W-32NO”, and “PROMOIS W-32R” (weight-average molecular weight: 400) manufactured by Seiwa Kasei Co., Ltd., “PROMOIS W-52”, “PROMOIS W-52P”, and “PROMOIS W-52Q” (weight-average molecular weight: 2000) manufactured by Seiwa Kasei Co., Ltd., “PHARCONIX CTP-F(BG)” (weight-average molecular weight: 5000) manufactured by ICHIMARU PHARCOS Co., Ltd., “NIPPI COLLAGEN-AFD”, “NIPPI COLLAGEN-FCP”, and “NIPPI COLLAGEN-FCP-G” (weight-average molecular weight: 3000 to 5000) manufactured by Nippi Inc., and “JELLYFISH COLLAGEN” manufactured by Javenech. The weight-average molecular weight of the low-molecular collagen is preferably approximately 200 to 20000, more preferably approximately 1000 to 10000, further preferably approximately 2000 to 5000.

In the present invention, in the case of using the collagens as the migration imparting agent and preparing this as an external skin preparation, the content of the collagens is preferably 0.00001% by weight or more, more preferably 0.0001% by weight or more, still more preferably 0.001% by weight or more, with respect to the total weight of the external skin preparation. Also, it is, for example, preferably 20% by weight or less, more preferably 10% by weight or less, further preferably 5% by weight or less, most preferably 3% by weight or less, with respect to the total weight of the external skin preparation from the viewpoint of suppressing reduction in usability.

In the present invention, in the case of using the oligopeptides as the migration imparting agent, it is preferable to use, among them, particularly, at least one selected from the group consisting of dipeptides, tripeptides, tetrapeptides and derivatives thereof, and it is more preferable to especially use tripeptides, tetrapeptides, and derivatives thereof. Among others, oligopeptides constituted by amino acids selected from glycine, histidine, lysine, threonine, and serine are preferable. Particularly, oligopeptides in which a terminal amino group or carboxyl group is further converted, for example, a terminal carboxyl group is esterified, oligopeptides complexed with metals, and the like are preferably used as derivatives of the oligopeptides. More specifically, copper tripeptide-1 (copper complex compound of tripeptide constituted by glycine, histidine, and lysine), caprooyl tetrapeptide-3 (reaction product of tetrapeptide constituted by lysine, threonine, and serine and caproic acid), and the like are preferably used.

The oligopeptides may also be used singly or in combinations of two or more.

In the present invention, in the case of using the oligopeptides as the migration imparting agent and preparing this as an external skin preparation, the content of the oligopeptides can be set to 0.000001% by weight or more and is preferably 0.00001% by weight or more, more preferably 0.0001% by weight or more, still more preferably 0.001% by weight or more, with respect to the total weight of the external skin preparation. Also, it is, for example, preferably 1% by weight or less, more preferably 0.5% by weight or less, further preferably 0.3% by weight or less, most preferably 0.1% by weight or less, with respect to the total weight of the external skin preparation.

Additional specific examples of the migration imparting agent used in the present invention include a combination of collagen and derivatives of oligopeptides. As the combination of collagen and oligopeptides, a combination of collagen, oligopeptide that has formed a derivative by reacting with a fatty acid (e.g., caproic acid or palmitic acid), and oligopeptide complexed with a metal is preferable, a combination of collagen, copper tripeptide-1 and caprooyl tetrapeptide-3 is more preferable, and a combination of jellyfish-derived collagen, copper tripeptide-1 and caprooyl tetrapeptide-3 is further preferable. Although the mixing ratio of each component in the combination of collagen (e.g., jellyfish-derived collagen), copper tripeptide-1 and caprooyl tetrapeptide-3 is not particularly limited, it is preferable to be 1:0.01 to 10:0.01 to 10 (based on weight), it is more preferable to be 1:0.03 to 5:0.03 to 5 (based on weight), and it is further preferable to be 1:0.05 to 3:0.05 to 3 (based on weight).

As a method for applying the migration imparting agent to the skin, for example, it is preferable to allow the migration imparting agent to be contained as a component in an external skin preparation (including a cosmetic), and apply the external skin preparation in a form appropriate for a dosage form to the skin.

Such an external skin preparation can be used in the form of an aqueous solution of purified water or pure water, or a mixed solution of a lower alcohol, a polyhydric alcohol, or the like and water. Also, a cream form in which an oil component and an aqueous component are emulsified can be prepared. Further, a jelly form, a predetermined shape made by solidification, or a granule or powder form can also be prepared.

It is preferable that a solidified, granule, or powder form should be dissolved in water or the like upon use and applied to the skin. On the other hand, a solution, emulsion, cream, lotion, paste, mousse, or gel form can be applied as it is to the skin. Furthermore, an aerosol or spray form can be used by spraying this from a spray-type container toward the skin.

Alternatively, a migration imparting agent-containing composition prepared in a liquid form, a cream form, or the like may be supported by a separately provided sheet base material. Furthermore, the migration imparting agent-containing composition itself may be molded into a sheet shape. The sheet-shaped external skin preparation thus obtained can be applied as a patch to the skin, thereby continuously supplying the migration imparting agent to the skin for a predetermined time.

Examples of optional components that can be used with the migration imparting agent in the external skin preparation include various components that are generally used as components for cosmetic compositions to be applied to the skin.

Examples thereof include anionic surfactants, cationic surfactants, ampholytic surfactants, silicone oil, waxes, alcohols, natural extracts, protein hydrolysates, germicides, anti-inflammatory agents, antiseptics, antioxidants, ultraviolet absorbers, pH adjusters, chelating agents, humectants, emulsifiers, vitamin agents, various whitening components, pigments, dyes, and fragrances.

According to the external skin preparation of the present invention, since a heretofore unknown special migration imparting agent that exerts a migration imparting effect on adipose-derived stem cells present below the dermal layer can be applied from the skin surface side to penetrate the dermal layer, adipose-derived stem cells migrate to the dermal layer side by the action of this migration imparting agent and become abundantly present in the dermal layer. Therefore, the adipose-derived stem cells are abundantly present in the dermal layer, and senescence or injury of fibroblasts which contribute to skin elasticity and firmness is ameliorated because they are replaced with new fibroblasts in very short cycles by the multilineage potential of the adipose-derived stem cells; thus skin elasticity and firmness are improved, and excellent cosmetic effects can be obtained.

Also, since the adipose-derived stem cells secrete, in themselves, factors associated with the construction of extracellular matrix (ECM) in the dermal layer and also have the effect of activating fibroblasts, fibroblasts themselves in the dermal layer are activated when the adipose-derived stem cells are abundantly present in the dermal layer; thus much better cosmetic effects can be obtained.

Furthermore, it is known that the adipose-derived stem cells produce, in themselves, ECM-associated factors such as type I collagen, MMP1 (matrix metalloproteinase 1: an enzyme involved in cleavage and reconstruction of collagen), hyaluronic acid, and TIMP1 (tissue inhibitor of metalloproteinase: an enzyme inhibiting MMP1 and adjusting balance). From this, the state within the dermal layer is kept better when the adipose-derived stem cells are abundantly present in the dermal layer; thus excellent cosmetic effects can also be obtained.

Accordingly, the present invention proposes not only a cosmetic method comprising allowing adipose-derived stem cells to migrate to the dermal layer by using a migration imparting agent, but also a cosmetic method comprising allowing adipose-derived stem cells and fibroblasts to coexist by directly introducing the adipose-derived stem cells to the dermal layer by penetration, implantation, or the like, thereby producing a skin condition improving effect.

In such a cosmetic method, the penetration or implantation of the adipose-derived stein cells can be performed by, for example, penetration by combined use with a transdermal absorption promoting agent, penetration by electroporation using electric pulse, or implantation by injection using a syringe. In general, it is preferable to allow both the cells to coexist by a method of applying the migration imparting agent as described in the present specification to the skin, from the viewpoint that the fibroblasts and the adipose-derived stem cells can be allowed to coexist conveniently and efficiently.

Some migration imparting agents used in the present invention not only exert a migration imparting agent effect but exert a growth promoting effect, on adipose-derived stem cells. Accordingly, by using such a migration imparting agent, not only are adipose-derived stem cells in the fat layer allowed to migrate to the dermal layer, but the growth of adipose-derived stem cells themselves can be promoted, thereby more remarkably increasing the number of adipose-derived stem cells in the dermal layer; thus excellent cosmetic effects can be obtained.

Incidentally, in the migration imparting agent used in the present invention, examples of substances known to exert not only a migration imparting agent effect but a growth promoting effect on adipose-derived stein cells include the aforementioned collagens, oligopeptides, and oligopeptide derivatives.

The present invention mentioned above can also be interpreted as an external skin preparation for improvement in skin condition, the external skin preparation comprising a migration imparting agent for adipose-derived stem cells. The present invention can also be interpreted as an external skin preparation for use in improvement in skin condition, the external skin preparation comprising a migration imparting agent for adipose-derived stem cells. The present invention can be further interpreted as use of a migration imparting agent for adipose-derived stem cells in production of an external skin preparation for use in improvement in skin condition. Specific forms of each element in these embodiments are as described above.

The present invention further provides a method of screening for a component for use in a cosmetic method for improving a skin condition, the screening method comprising evaluating migratory activity of adipose-derived stem cells by using a prepared medium containing a sample.

In the screening method of the present invention, the step of evaluating migratory activity of adipose-derived stem cells can be performed by an arbitrary cell migration assay approach. It can be carried out by, for example, a method comprising: respectively providing a prepared medium containing a sample and a standard medium not containing the sample; culturing adipose-derived stem cells for a predetermined period in Transwell by using the prepared medium or the standard medium; and selecting the component for use in a cosmetic method for improving a skin condition by using, as an index, higher migratory activity of the adipose-derived stem cells when the prepared medium is used than the migratory activity of the adipose-derived stem cells when the standard medium is used. The cell migration assay approach does not mimic a wound condition by performing scratch treatment or the like and therefore, does not evaluate the migratory activity of adipose-derived stem cells under influence of factors or the like induced by wounding. Accordingly, in other words, migratory activity in the case where the skin condition is a condition of the non-wound skin is evaluated. Thus, the cell migration assay approach can also be carried out as a method of screening for a component improving a condition of the non-wound skin. The screening method can also be carried out by measuring in advance migratory activity of adipose-derived stem cells when the standard medium not containing the sample is used, and comparing the reference value with migratory activity of adipose-derived stem cells when the prepared medium containing a sample is used.

In one embodiment, the screening method of the present invention can be performed by, for example, a two-layer culture method. Specifically, it can be carried out by, for example, a method comprising: providing a first two-layer culture device (plate, etc.), and inoculating adipose-derived stem cells to upper wells thereof while supplying a prepared medium containing a sample to lower wells thereof; providing a second two-layer culture device, and inoculating adipose-derived stem cells to upper wells thereof while supplying a standard medium not containing the sample to lower wells thereof, performing culture for a predetermined period in the first and second two-layer culture devices; and measuring, by an arbitrary cell count measurement approach, the number of adipose-derived stem cells that have moved from the upper side to the lower side via a porous filter separating between the upper wells and the lower wells in each of the two-layer culture devices, and evaluating migration imparting agent properties of the sample by using, as an index, the rate of increase of the number of adipose-derived stem cells on the lower side of the first two-layer culture device with respect to the number of adipose-derived stem cells on the lower side of the second two-layer culture device. In the screening method of the present invention, for example, Boyden chambers, scaffolds (porous three-dimensional culture scaffolds, etc.), or Zigmond chambers can be used as the two-layer culture devices. In the screening method, examples of the cell count measurement approach include a method of evaluating enhancement of fluorescence emission by using Calcein-AM or the like, and a method of measuring absorbance by using a crystal violet reagent, a MTT reagent, a WST reagent, or the like.

In the screening method of the present invention, it is preferable that the period of the step of culturing the adipose-derived stem cells should be a shorter period than the doubling time of these cells. For example, the period of the step of culturing the adipose-derived stem cells is preferably shorter than 24 hours, more preferably 22 hours or shorter, still more preferably 20 hours or shorter.

The screening method of the present invention may further comprise evaluating growth promoting activity against adipose-derived stein cells. By including such a step, a cosmetic component that can not only exert a migration imparting effect on adipose-derived stem cells to the dermal layer but exert the effect of promoting growth of adipose-derived stem cells themselves in the dermal layer, and is much more effective for improvement in skin condition can be selected.

The step of evaluating growth promoting activity against adipose-derived stem cells as described above can be performed by an arbitrary cell growth assay approach. For example, adipose-derived stem cells are inoculated to each of a prepared medium containing a sample and a standard medium not containing the sample; after culture for a predetermined period, the number of adipose-derived stem cells in each medium is measured; and the growth promoting activity against adipose-derived stem cells can be evaluated as being present in the case where the number of cells in the prepared medium containing a sample is significantly increased as compared with the standard medium not containing the sample. The measurement of the number of cells can be performed by an arbitrary cell count measurement approach known in the art as mentioned above, and can be performed by, for example, the measurement of absorbance using a WST reagent or the like. The step of evaluating growth promoting activity against adipose-derived stem cells can also be carried out by measuring in advance the number of adipose-derived stem cells when the standard medium not containing the sample is used, and comparing the reference value with the number of adipose-derived stem cells when the prepared medium containing a sample is used.

In the step of evaluating growth promoting activity against adipose-derived stem cells, it is preferable that the period of the step of culturing the adipose-derived stein cells should be a longer period than the doubling time of these cells. For example, the period of the step of culturing the adipose-derived stem cells is preferably 30 hours or longer, more preferably 50 hours or longer, still more preferably 70 hours or longer.

The screening method of the present invention is used for selecting, for example, a component improving a condition of the non-wound skin (i.e., the healthy skin) which is not a wound site. Such a component improving a condition of a non-wound site can be used as a cosmetic component (component for use in cosmetics, etc.) ameliorating a condition of the healthy skin, not as a medicament for use in treatment of wound.

In one embodiment, the screening method of the present invention may be a method of screening for a component improving a condition of the non-wound skin by using a three-dimensional skin model (non-wound skin model) in which a labeled adipose-derived stem cell layer, a dermal model layer and an epidermal keratinocyte layer are stacked in this order. The component selected by the screening method can be used in a cosmetic method for improving a condition of the non-wound skin. The screening method according to the present embodiment comprises: providing a three-dimensional skin model in which a labeled adipose-derived stem cell layer, a dermal model layer and an epidermal keratinocyte layer are stacked in this order; applying a sample to the skin model, followed by culture for a predetermined period; measuring the number of adipose-derived stem cells that have migrated to the dermal model layer and/or the epidermal keratinocyte layer by using the label in the skin model after the culture; and selecting the component improving a condition of the non-wound skin by using, as an index, the number of adipose-derived stem cells that have migrated to the dermal model layer and/or the epidermal keratinocyte layer. The non-wound skin model can be prepared by, for example, a method comprising: inoculating labeled adipose-derived stem cells to the bottom surface of upper wells of a two-layer culture device, followed by culture to form a labeled adipose-derived stem cell layer; stacking a dermal model layer (e.g., a layer consisting of a collagen gel mixed with fibroblasts) on the labeled adipose-derived stem cell layer; and inoculating epidermal keratinocytes onto the dermal model layer, followed by culture to form an epidermal keratinocyte layer. Examples of the method for applying a sample to the non-wound skin model can include a method of applying a sample onto the epidermal keratinocyte layer by addition as well as a method of using addition of a sample and ultrasonic introduction or ionic introduction in combination, a method of applying a transdermal absorption promoting agent with a sample by addition, and a method of adding a sample into lower wells of a two-layer culture device and applying it via a porous filter. The application of a sample to the non-wound skin model can also be carried out, for example, by allowing the sample to be contained in advance in a collagen gel or the like for the dermal model layer in the course of preparation of the non-wound skin model. The culture period after the application of the sample is, for example, preferably 7 days or shorter, more preferably 5 days or shorter, still more preferably 3 days or shorter. The step of measuring the number of adipose-derived stem cells that have migrated to the dermal model layer and/or the epidermal keratinocyte layer by using the label in the skin model after the culture can be carried out, for example, by slicing (sectioning) the non-wound skin model in a lengthwise direction or the like, and counting the labels on adipose-derived stem cells that have migrated to the dermal model layer and/or the epidermal keratinocyte layer. Examples of the method for labeling the adipose-derived stem cells can include a method of expressing a fluorescent protein such as green fluorescent protein (GFP), yellow fluorescent protein (YFP), red fluorescent protein (RFP), or cyan fluorescent protein (CFP), and a method of using a cell labeling reagent (CELL TRACE series (manufactured by Thermo Fisher Scientific Inc.), etc.). The step of selecting the component improving a condition of the non-wound skin by using, as an index, the number of adipose-derived stem cells that have migrated to the dermal model layer and/or the epidermal keratinocyte layer can determine and select the component improving a condition of the non-wound skin in the case where the number of adipose-derived stem cells that have migrated to the dermal model layer and/or the epidermal keratinocyte layer when the sample is applied is larger than the number of adipose-derived stem cells that have migrated to the dermal model layer and/or the epidermal keratinocyte layer when the same operation is carried out without application of the sample.

In the present invention, the presence or absence of the migration imparting effect of the migration imparting agent on adipose-derived stem cells can be confirmed by, for example, the following test and criteria for determination.

<Confirmation Test and Criteria for Determination for Migration Imparting Effect>

A medium of human adipose-derived stem cells (manufactured by Kurabo Industries Ltd.; cultured to the 7th passage in STEMLIFE medium [provided by Lifeline Cell Technology]) was replaced with DMEM/F12 medium (manufactured by Life Technologies Corp.), followed by culture for additional 20 hours.

Meanwhile, a medium in which a sample to be examined for the presence or absence of the migration imparting effect was dissolved at a predetermined concentration was prepared by using 0.1% by volume of fetal bovine serum (nutritional source). Also, DMEM/F12 medium containing 0.1% by volume of fetal bovine serum and not containing the sample (blank) was provided.

Then, the prepared medium was supplied at 200 μL per well to lower wells of a 96-well Boyden chamber (manufactured by Corning Inc., model number: 3384) having a membrane pore size of 8 μm. Also, the human adipose-derived stem cells cultured for 20 hours in the DMEM/F12 medium were inoculated at 30000 cells per well to upper wells of the Boyden chamber and cultured at 37° C. for 20 hours under 5% by volume of CO₂.

1.2 μL/mL of Calcein-AM (fluorescent dye, manufactured by Molecular Probes/Thermo Fisher Scientific Inc.) dissolved at 1.66 mg/mL in DMSO (manufactured by Wako Pure Chemical Industries, Ltd.) was added to 10× cell dissociation solution (cell dissolution solution, manufactured by Trevigen Inc.) diluted 10-fold with sterilized water to thereby prepare an index solution for fluorescence emission having the correlation between the number of human adipose-derived stem cells and the strength of fluorescence emission.

The media in the upper and lower wells were taken out of the Boyden chamber and washed with PBS(−) (manufactured by Dojindo Laboratories), and then, the index solution for fluorescence emission was supplied at 100 μL per well to the lower wells and incubated at 37° C. for 1 hour under 5% by volume of CO₂ in the dark. Then, the index solution in the lower wells after the incubation was transferred to a 96-well plate (manufactured by Greiner Bio One International GmbH) having black walls and a transparent bottom, and fluorescence intensity (excitation wavelength: 485 nm, detection wavelength: 520 nm) was measured. Each test was conducted in triplicate (n=3).

Specifically, since there is a correlation between the number of human adipose-derived stem cells and the strength of fluorescence emission as mentioned above, the presence or absence of the migration imparting effect was confirmed by determining increase or decrease in the fluorescence intensity of the lower wells by using the fluorescence intensity obtained by the assay of the blank as a reference (100%). As for the determination of the presence or absence of migration, the “migration imparting effect was present” was determined provided that the rate of stem cell migration (rate of increase with respect to the blank) was 5% or more (score + or more) according to the score shown in Table 1 below.

TABLE 1

| Rate of stem cell migration (%) | Score |
|---|---|
| Less than 95 | − |
| 95 or more and less than 105 | ± |
| 105 or more and less than 120 | + |
| 120 or more and less than 130 | ++ |
| 130 or more and less than 140 | +++ |
| 140 or more | ++++ |

<Confirmation Test and Criteria for Determination for Growth Promoting Effect>

A medium in which a sample to be examined for the presence or absence of the migration imparting effect was dissolved at a predetermined concentration was prepared by using DMEM/F12 medium (manufactured by Life Technologies Corp.) containing 0.1% by volume of fetal bovine serum. Also, DMEM/F12 medium containing 0.1% by volume of fetal bovine serum unsupplemented with the sample (blank) was provided.

Then, human adipose-derived stem cells (manufactured by Kurabo Industries Ltd.; cultured to the 5th passage in STEMLIFE medium [provided by Lifeline Cell Technology]) were inoculated at 3000 cells per well with the prepared medium to a 96-well plate and cultured at 37° C. for 72 hours under 5% by volume of CO₂, and then, respiratory activity of the cells was measured as an index for whether or not the cells were activated, by the WST-8 method using Cell Counting Kit-8 (manufactured by Dojindo Laboratories). Specifically, after the human adipose-derived stem cells were cultured for 72 hours, the medium of each well was replaced with DMEM/F12 medium containing Cell Counting Kit-8, which was then incubated at 37° C. for 4 hours under 5% by volume of CO₂ in the dark, and then, absorbance at a wavelength of 450 nm (control wavelength: 630 nm) was measured. Each test was conducted in triplicate (n=3).

Specifically, the degree of the respiratory activity (proportional to the number of cells) was measured as the absorbance (wavelength: 450 nm) of the solution by using the WST-8 reagent. Then, the presence or absence of the growth promoting effect was confirmed by determining increase or decrease in the absorbance of the sample-containing prepared medium by using the absorbance obtained by the assay of the blank medium as a reference (100%). As for the determination of the presence or absence of growth promotion, the “stem cell growth promoting effect was present” was determined provided that the rate of stem cell growth (rate of growth with respect to the blank) was 5% or more (score + or more) according to the score shown in Table 2 below.

TABLE 2

| Rate of stem cell growth (%) | Score |
|---|---|
| Less than 95 | − |
| 95 or more and less than 105 | ± |
| 105 or more and less than 120 | + |
| 120 or more and less than 130 | ++ |
| 130 or more and less than 140 | +++ |
| 140 or more | ++++ |

<Migration Imparting Effect and Growth Promoting Effect>

It is known that the doubling time of the present cells is usually 24 hours or longer, and in the confirmation test for the migration imparting effect, the human adipose-derived stem cells are taken out 20 hours after inoculation and washed, followed by the measurement of the strength of fluorescence emission. Accordingly, in this test, the growth effect of the human adipose-derived stem cells themselves is not included, and only increase in cells by migration is evaluated. On the other hand, in the confirmation test for the growth promoting effect, absorbance 72 hours after inoculation of the human adipose-derived stein cells is measured, and increase in cells by self-growth is simply evaluated.

### EXAMPLES

Hereinafter, Examples of the present invention will be described in detail together with Comparative Examples.

### Examples 1 to 5 and Comparative Example 1

For four migration imparting agents used in the cosmetic method of the present invention, jellyfish-derived collagen (JELLYFISH COLLAGEN, manufactured by Arysta LifeScience Corp.), copper tripeptide-1 (COPPER PEPTIDE, manufactured by Arysta LifeScience Corp.), caprooyl tetrapeptide-3 (CHRONOLINE, manufactured by Arysta LifeScience Corp.), and dipropylene glycol, the confirmation tests for their migration imparting effects and growth promoting effects were conducted according to the methods described above. Also, the same tests were conducted on lavender oil as Comparative Example 1. The results are shown in Tables 3 to 8 below. The applied concentrations of the migration imparting agents are as respectively shown in Tables 3 to 8 below.

TABLE 3
Example 1

| | | Applied | Ratio to | |
|---|---|---|---|---|
| Substance | | concentration | blank | |
| evaluated | Evaluation item | (mg/mL) | (%) | Evaluation |
| Jellyfish- | Migration | 0.1 | 110 | + |
| derived | imparting effect | 1.0 | 128 | ++ |
| collagen | Growth | 0.1 | 113 | + |
| | promoting effect | 1.0 | 122 | ++ |

TABLE 4
Example 2

| | | Applied | Ratio to | |
|---|---|---|---|---|
| Substance | | concentration | blank | |
| evaluated | Evaluation item | (mg/mL) | (%) | Evaluation |
| Copper | Migration | 0.001 | 130 | +++ |
| tripeptide-1 | imparting effect | 0.01 | 125 | ++ |
| | | 0.1 | 131 | +++ |
| | Growth | 0.01 | 130 | +++ |
| | promoting effect | 0.1 | 160 | ++++ |

TABLE 5
Example 3

| | | Applied | Ratio to | |
|---|---|---|---|---|
| Substance | | concentration | blank | |
| evaluated | Evaluation item | (mg/mL) | (%) | Evaluation |
| Caprooyl | Migration | 0.01 | 109 | + |
| tetrapeptide-3 | imparting effect | 0.1 | 133 | +++ |
| | | 1.0 | 132 | +++ |
| | Growth | 0.01 | 120 | ++ |
| | promoting effect | 0.1 | 123 | ++ |
| | | 1.0 | 117 | + |

TABLE 6
Example 4

| | | Applied | Ratio to | |
|---|---|---|---|---|
| Substance | | concentration | blank | |
| evaluated | Evaluation item | (mg/mL) | (%) | Evaluation |
| Dipropylene | Migration | 0.000001 | 109 | + |
| glycol | imparting effect | 0.00001 | 125 | ++ |
| | | 0.0001 | 150 | ++++ |
| | Growth | 0.000001 | 115 | + |
| | promoting effect | 0.00001 | 108 | + |
| | | 0.0001 | 117 | + |

TABLE 7
Example 5

| | | Applied | Ratio to | |
|---|---|---|---|---|
| Substance | | concentration | blank | |
| evaluated | Evaluation item | (mg/mL) | (%) | Evaluation |
| Complex A* | Migration | 0.0003 | 112 | + |
| | imparting effect | 0.003 | 122 | ++ |
| | | 0.03 | 162 | ++++ |

*Complex A is a substance containing jellyfish-derived collagen, copper tripeptide-1, and caprooyl tetrapeptide-3 at a ratio of 1:1:1 (based on weight).

TABLE 8
Comparative Example 1

| | | Applied | Ratio to | |
|---|---|---|---|---|
| Substance | | concentration | blank | |
| evaluated | Evaluation item | (mg/mL) | (%) | Evaluation |
| Lavender oil | Migration | 0.000001 | 71 | − |
| | imparting effect | 0.005 | 78 | − |
| | | 0.01 | 79 | − |
| | Growth | 0.002 | 103 | ± |
| | promoting effect | 0.005 | 117 | + |
| | | 0.01 | 102 | ± |

From the results described above, it is evident for Examples 1 to 5 that human adipose-derived stem cells migrated from the upper side to the lower side of the membrane by the migration imparting agent used. Accordingly, provided that an external skin preparation using this migration imparting agent is applied to the skin, excellent cosmetic effects are obtained. By contrast, the lavender oil used in Comparative Example 1 exhibits only the growth promoting effect and does not have the migration imparting effect. Accordingly, excellent cosmetic effects as in Examples 1 to 5 cannot be obtained.

### Example 6

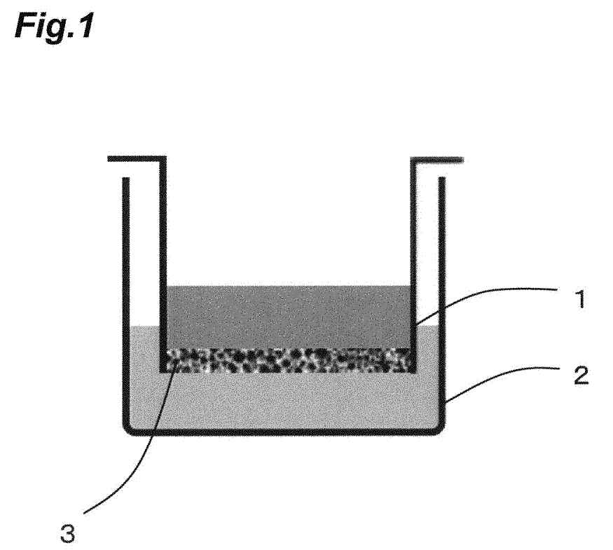

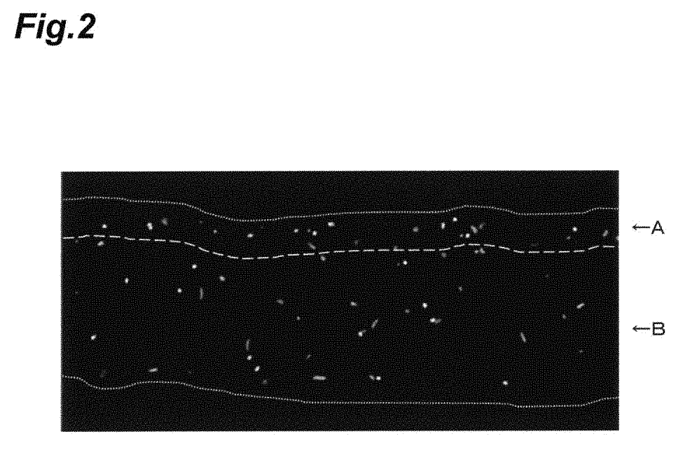

As shown in FIG. 1, a porous three-dimensional culture scaffold (ALVETEX Scaffold 12 well inserts, manufactured by ReproCELL Inc.) prepared by integrating upper well 1 with scaffold 3 was coated with type I collagen (rat tail-derived, manufactured by Life Technologies Corp., concentration: 0.8 mg/mL) (assuming a dermal environment) and installed in lower wells 2 consisting of a 12-well plate (manufactured by Corning Inc.), and human adipose-derived stein cells were inoculated. Then, a medium containing 0.3 mg/mL of the evaluated substance (containing jellyfish-derived collagen, copper tripeptide-1, and caprooyl tetrapeptide-3 at a ratio of 1:1:1) used in Example 5 was added to the lower wells 2 and cultured for 5 days (scaffold migration test). After the culture, the tissue was fixed in 4% by weight of a paraformaldehyde solution and embedded in paraffin. The tissue was sliced in the vertical direction into a section having a thickness of 10 μm by using a microtome, and the nuclei of human adipose-derived stem cells present in the cut face were visualized blue by Hoechst 33258 staining. This cut face was photographed under a fluorescence microscope (IX71, manufactured by Olympus Corp., magnification: ×10), and the obtained scaffold section photograph is shown in FIG. 2.

### Comparative Example 2

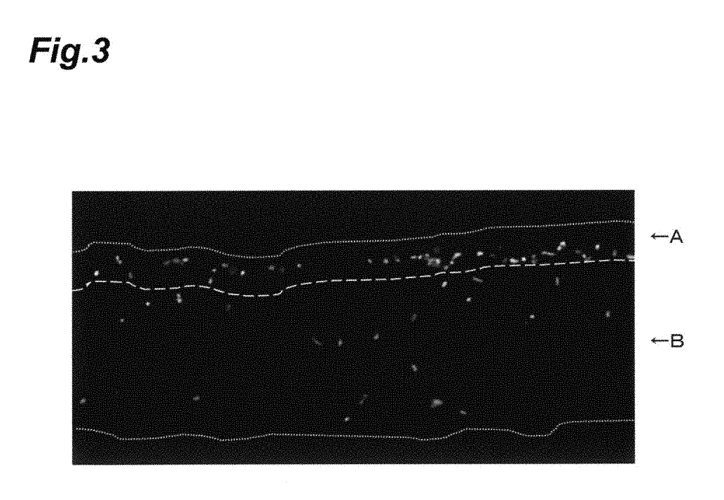

In the same scaffold migration test as above, one containing no evaluated substance was used as a medium of the lower wells 2 (see FIG. 1). The other procedures were performed in the same way as in Example 6 described above to obtain a scaffold section photograph. This is shown in FIG. 3.

From the comparison between FIGS. 2 and 3, it is evident that human adipose-derived stem cells entered the scaffold 3 to a large extent in the medium of Example 6 containing the evaluated substance (containing the three evaluated substances used in Examples 1 to 3 at a ratio of 1:1:1), as compared with Comparative Example 2 not containing this. Cells that moved to a thickness portion (indicated by B in FIGS. 2 and 3) below a thickness portion of approximately ¼ (indicated by A in FIGS. 2 and 3) from the top of the scaffold 3 was defined as “cells that migrated”, and the ratio of “the number of migratory cells/the total number of cells” is shown in Table 9 below. Cell counting was performed by using analytical software ImageJ. This result supports the promotion of migration of human adipose-derived stem cells by the three evaluated substances contained in the medium of Example 6.

TABLE 9

| | The number of | Total number of | Ratio of migratory |
|---|---|---|---|
| | migratory cell | cell | cell |
| Example 6 | 49 | 86 | 57% |
| Comparative | 31 | 88 | 35% |
| Example 2 | | | |

The following confirmation tests were further conducted as to the significance of abundant presence of adipose-derived stem cells in the dermal layer.

[Confirmation Test 1: Comparison of Amount of Type I Collagen Produced Between Human Adipose-Derived Stem Cell and Human Dermal Fibroblast]

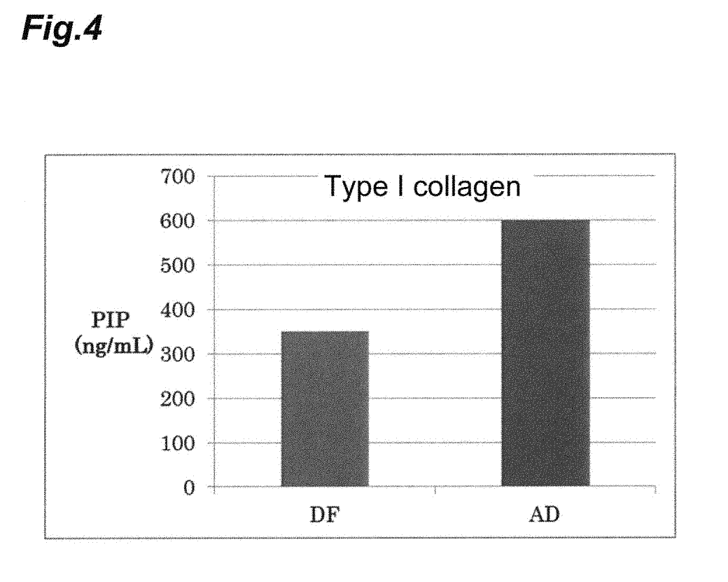

Human dermal fibroblasts (manufactured by Kurabo Industries Ltd.) and human adipose-derived stem cells (Lifeline Cell Technology) were cultured to the 4th passages in environments recommended by the manufacturers, and respectively inoculated at 1.4×10⁶ cells to 100 mm Petri dishes. After overnight incubation, the media were replaced with DMEM/F12 containing 0.2% by volume of fetal bovine serum, followed by further culture. 48 hours after the medium replacement, the culture supernatant of the human dermal fibroblasts (DF) and the culture supernatant of the human adipose-derived stem cells (AD) were recovered, and PIP concentrations in the culture supernatants DF and AD were determined by ELISA (using PIP EIA kit: MK101 manufactured by Takara Bio Inc.). The PIP is a fragment peptide that is liberated by cleavage when type I procollagen, which is a type I collagen precursor, becomes mature type I collagen, and correlates with the amount of type I collagen produced. Accordingly, as the PIP concentration is higher, the amount of type I collagen produced can be evaluated as being larger. The results are as shown in FIG. 4, and it is evident that the amount of type I collagen produced is remarkably larger in the human adipose-derived stem cells as compared with the human dermal fibroblasts.

[Confirmation Test 2: Comparison of Amount of MMP1 Produced and Activity Between Human Adipose-Derived Stem Cell and Human Dermal Fibroblast]

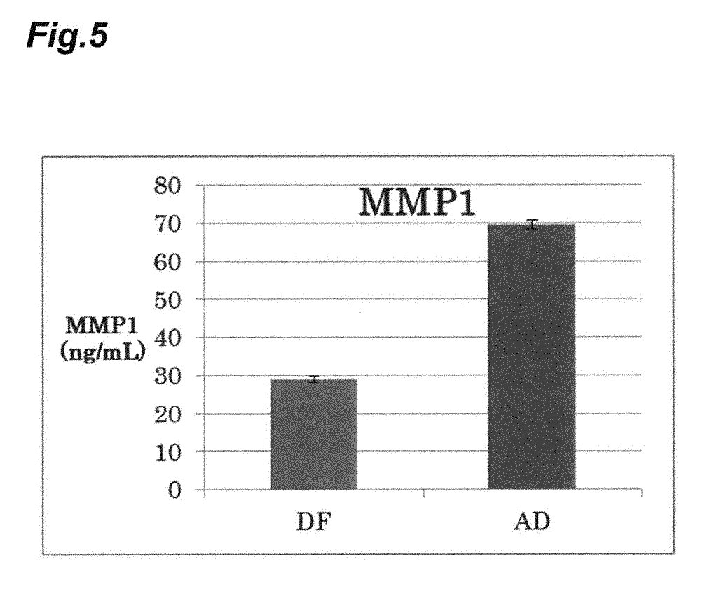

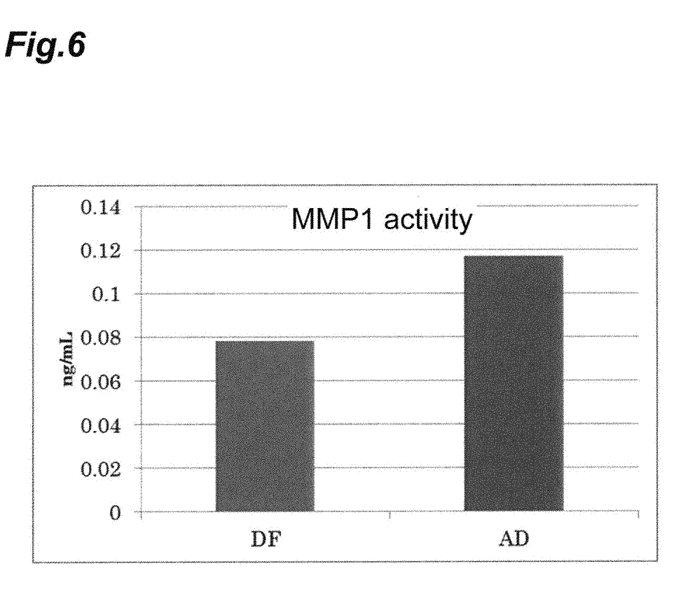

In the same way as in confirmation test 1 described above, the culture supernatant of the human dermal fibroblasts (DF) and the culture supernatant of the human adipose-derived stem cells (AD) were recovered. Then, MMP1 concentrations in the culture supernatants DF and AD were determined by ELISA (using total MMP1 ELISA kit: DY901 manufactured by R&D Systems, Inc.). Also, MMP1 activity in the culture supernatants DF and AD was measured in a system degrading fluorescently labeled collagen (using human active MMP1 Fluorokine E kit: F1M00 manufactured by R&D Systems, Inc.). The results are as shown in FIGS. 5 and 6, and it is evident that both the amount of MMP1 produced and the activity are larger (higher) in the human adipose-derived stem cells as compared with the human dermal fibroblasts.

[Confirmation Test 3: Comparison of Amount of Hyaluronic Acid Produced Between Human Adipose-Derived Stem Cell and Human Dermal Fibroblast]

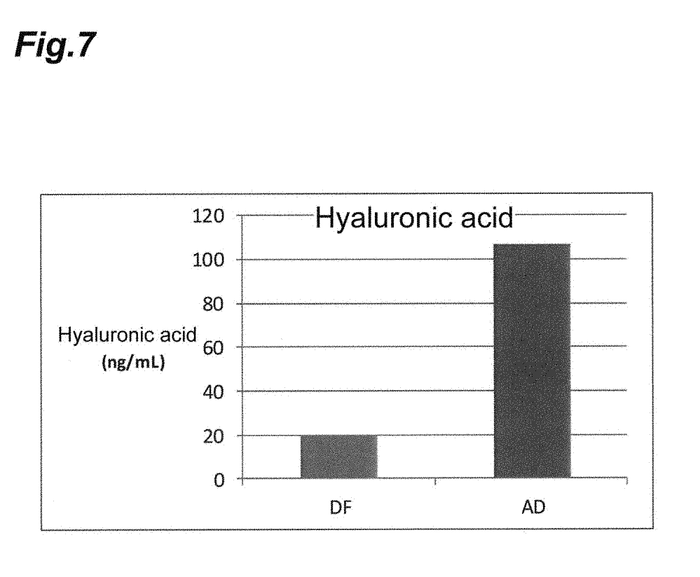

In the same way as in confirmation test 1 described above, the culture supernatant of the human dermal fibroblasts (DF) and the culture supernatant of the human adipose-derived stem cells (AD) were recovered. Then, hyaluronic acid concentrations in the culture supernatants DF and AD were determined by ELISA (using Hyaluronan Quantikine ELISA kit: DHYAL0 manufactured by R&D Systems, Inc.). The results are as shown in FIG. 7, and it is evident that the amount of hyaluronic acid produced is remarkably larger in the human adipose-derived stem cells as compared with the human dermal fibroblasts.

[Confirmation Test 4: Comparison of Amount of TIMP1 Produced Between Human Adipose-Derived Stem Cell and Human Dermal Fibroblast]

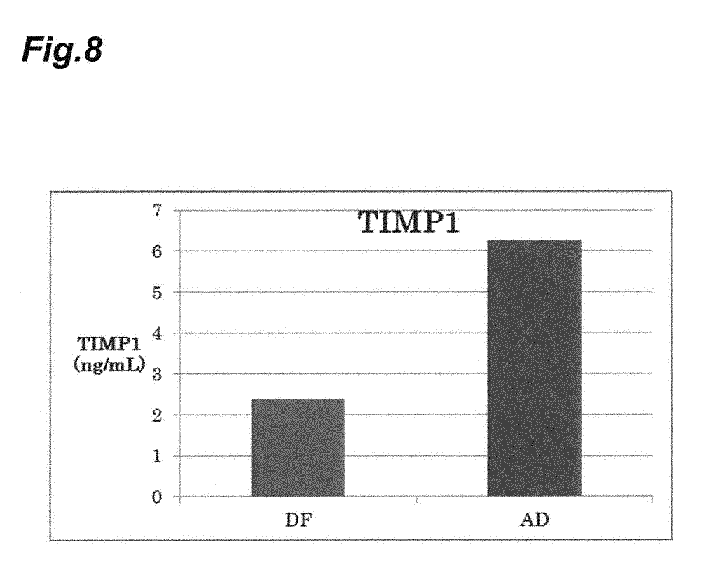

In the same way as in confirmation test 1 described above, the culture supernatant of the human dermal fibroblasts (DF) and the culture supernatant of the human adipose-derived stem cells (AD) were recovered. Then, TIMP1 concentrations in the culture supernatants DF and AD were determined by ELISA (using Human TIMP-1 Quantikine ELISA Kit: DTM100 manufactured by R&D Systems, Inc.). The results are as shown in FIG. 8, and it is evident that the amount of TIMP1 produced is remarkably larger in the human adipose-derived stem cells as compared with the human dermal fibroblasts.

[Confirmation Test 5: Amount of TIMP1 Produced in Human Dermal Fibroblast when Human Adipose-Derived Stem Cell and Human Dermal Fibroblast were Directly Co-Cultured]

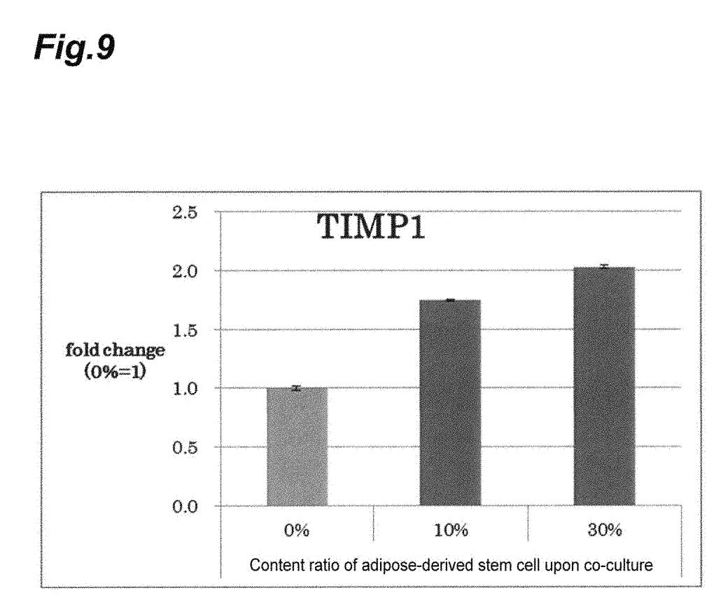

Human dermal fibroblasts (manufactured by Kurabo Industries Ltd.) were cultured in an environment recommended by the manufacturer, and stained with 2.5 μM Cell Trace Violet (manufactured by Thermo Fisher Scientific Inc.; C34557). The human dermal fibroblasts and human adipose-derived stem cells (manufactured by Lifeline Cell Technology) cultured in an environment recommended by the manufacturer were inoculated with varying content ratios of the human adipose-derived stem cells to 100 mm Petri dishes such that the number of cells was equally a total of 9×10⁵ cells to prepare three Petri dishes having a human adipose-derived stem cell content (ratio of the number of human adipose-derived stem cells to the total number of cells) of 0%, 10%, or 30%. Then, these were cultured for 72 hours, and then, only human dermal fibroblasts stained with Cell Trace Violet were isolated by using a cell sorter and inoculated at 2×10⁵ cells to 6-well plates. On the next day, the media were replaced with DMEM/F12 containing 0.2% by volume of fetal bovine serum, 48 hours thereafter, the culture supernatants were recovered, and TIMP1 concentrations in the culture supernatants were determined by ELISA (using Human TIMP-1 Quantikine ELISA Kit: DTM100 manufactured by R&D Systems, Inc.). The results are as shown in FIG. 9, and it is evident that the amount of TIMP1 produced in the human dermal fibroblasts is increased by direct co-culture with the human adipose-derived stem cells.

[Confirmation Test 6: Amount of Hyaluronic Acid Produced in Human Dermal Fibroblast when Human Adipose-Derived Stem Cell and Human Dermal Fibroblast were Directly Co-Cultured]

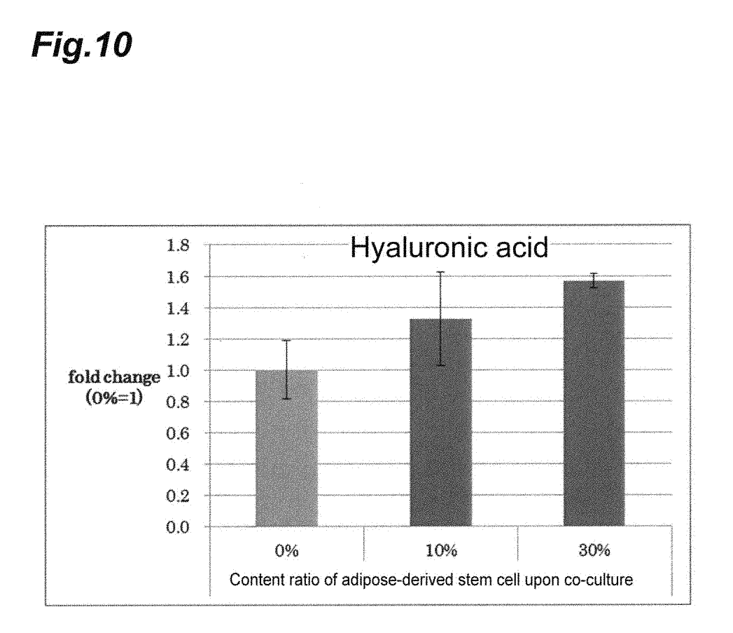

In the same way as in confirmation test 5 described above, the human adipose-derived stem cells and the human dermal fibroblasts were directly co-cultured to thereby obtain three culture supernatants differing in the content of the human adipose-derived stem cells. Hyaluronic acid concentrations in these culture supernatants were determined by ELISA (using Hyaluronan Quantikine ELISA kit: DHYAL0 manufactured by R&D Systems, Inc.). The results are as shown in FIG. 10, and it is evident that the amount of hyaluronic acid produced in the human dermal fibroblasts is increased by direct co-culture with the human adipose-derived stem cells.

[Confirmation Test 7: Amount of Type I Collagen Produced in Human Dermal Fibroblast when Human Adipose-Derived Stem Cell and Human Dermal Fibroblast were Directly Co-Cultured]

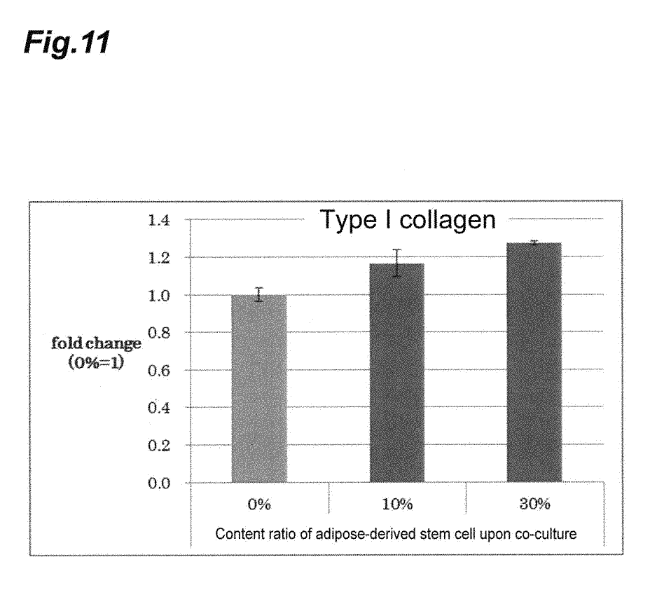

In the same way as in confirmation tests 5 and 6 described above, the human adipose-derived stem cells and the human dermal fibroblasts were directly co-cultured to thereby obtain three culture supernatants differing in the content of the human adipose-derived stem cells. Type I collage concentrations in these culture supernatants were determined by ELISA (using PIP EIA kit: MK101 manufactured by Takara Bio Inc.). The results are as shown in FIG. 11, and it is evident that the amount of type I collagen produced in the human dermal fibroblasts is increased by direct co-culture with the human adipose-derived stem cells.

[Confirmation Test 8: Comparison of Gene Expression Level of Extracellular Matrix Between Human Adipose-Derived Stem Cell and Human Dermal Fibroblast]

Since confirmation tests 1 to 7 described above made confirmation at the level of the amount of protein or polysaccharide produced in cells, whether or not human adipose-derived stem cells (manufactured by Lifeline Cell Technology) were also superior in gene expression level to human dermal fibroblasts (manufactured by Kurabo Industries Ltd.) was evaluated by analyzing the gene expression levels of extracellular matrix-associated factors in the respective cells using a microarray. When the gene expression levels in the human dermal fibroblasts were defined as 1, the gene expression levels in the human adipose-derived stem cells were converted to logarithmic values with a base of 2, and consequently, the results as shown in Table 10 below were obtained. Thus, it is evident that the human adipose-derived stem cells, as compared with the human dermal fibroblasts, also highly express MMP1, TIMP1, and HAS2 (hyaluronan synthase 2) at the gene level.

TABLE 10
Gene expression level in stem cell*

| | |
|---|---|
| MMP1 | 1.66 |
| TIMP1 | 2.46 |
| HAS2 | 3.06 |

*The expression ratio in stem cells with respect to the gene expression level in fibroblasts defined as 1 is indicated by a logarithmic value with a base of 2.

### INDUSTRIAL APPLICABILITY

The present invention can be widely utilized as a cosmetic method for improving a skin condition by using adipose-derived stem cells, an external skin preparation for use therein, a migration imparting agent, and a method of screening for a component for use in a cosmetic method for improving a skin condition.

### REFERENCE SIGNS LIST

1 . . . Upper well, 2 . . . Lower well, and 3 . . . Scaffold.

1. A cosmetic method comprising applying the topical skin preparation of claim 9 to the skin, thereby allowing adipose-derived stem cells to migrate to the dermal layer and improving skin condition.

2-7. (canceled)

8. The cosmetic method according to claim 1, wherein the migration imparting agent comprises a combination of copper tripeptide-1 and caprooyl tetrapeptide-3.

9. A topical skin preparation comprising a migration imparting agent allowing adipose-derived stem cells to migrate to the dermal layer by application to the skin, wherein the migration imparting agent comprises at least one oligopeptide complexed with a metal.

10. (canceled)

11. A method of screening for a component for use in a cosmetic method for improving a skin condition, the screening method comprising
evaluating migratory activity of adipose-derived stem cells by using a prepared medium containing a sample, wherein the migratory activity indicates skin condition improvement.

12. The method according to claim 11, wherein the skin condition is a condition of skin with no wound.

13. The cosmetic method according to claim 1, wherein the applying step allows fibroblasts and adipose-derived stem cells to coexist in the dermal layer, thereby improving skin condition.

14. The cosmetic method according to claim 13, wherein the fibroblasts and the adipose-derived stem cells are allowed to coexist by migration, implantation, or penetration of the adipose-derived stem cells.

15. The cosmetic method according to claim 8, further comprising applying at least one agent selected from the group consisting of dipropylene glycol, 1,2-butylene glycol, 1,3-butylene glycol, 1,4-butanediol, 1,3-butanediol, propylene glycol, isopropylene glycol, 1,3-propanediol, isoprene glycol, 1,2-pentanediol, hexylene glycol, 1,2-hexanediol, 1,6-hexanediol, glycerin, diglycerin, triglycerin, xylitol, galactitol, sorbitol, mannitol, ethylene glycol and diethylene glycol, to the skin.

16. The cosmetic method according to claim 1, wherein the migration imparting agent comprises a combination of copper tripeptide-1 and caprooyl tetrapeptide-3.

17. The cosmetic method according to claim 16, wherein the improving skin condition is based on improving skin elasticity and firmness.

18. The cosmetic method according to claim 16, wherein the improving skin condition is based on at least one process selected from the group consisting of increasing amount of type I collagen produced in the human adipose-derived stem cells, increasing amount of MMP1 produced in the human adipose-derived stem cells, increasing activity of MMP1 produced in the human adipose-derived stem cells, and increasing amount of TIMP1 produced in the human adipose-derived stem cells.

19. The cosmetic method according to claim 13, wherein the improving skin condition is based on increasing amount of TIMP1 produced in the human adipose-derived stem cells.

20. The cosmetic method according to claim 16, wherein the mixing ratio of each component in the combination of collagen, copper tripeptide-1 and caprooyl tetrapeptide-3 is 1:0.01 to 10:0.01 to 10, based on weight.

21. The topical skin preparation according to claim 9, wherein the content of the oligopeptides is 0.000001% by weight or more and 1% by weight or less with respect to the total weight of the topical skin preparation.

22. The topical skin preparation according to claim 9, wherein the migration imparting agent comprises a combination of collagen, copper tripeptide-1 and caprooyl tetrapeptide-3.
